(19) United States
(12) Patent Application Publication
(10) Pub. No.: US 2023/0142775 A1
(43) Pub. Date: May 11, 2023

(54) INFORMATION PROCESSING APPARATUS, INFORMATION PROCESSING METHOD, AND STORAGE MEDIUM

(71) Applicant: CANON KABUSHIKI KAISHA, Tokyo (JP)

(72) Inventor: Toru Kikuchi, Tokyo (JP)

(21) Appl. No.: 17/979,248

(22) Filed: Nov. 2, 2022

(30) Foreign Application Priority Data
Nov. 11, 2021 (JP) 2021-184222

(51) Int. Cl. G06T 7/00; G06T 7/70

(52) U.S. Cl. CPC G06T 7/0012; G06T 7/70; G06T 2207/30096

(57) ABSTRACT

An information processing apparatus comprises: an operating state obtaining unit configured to obtain information representing an operating state of detection processing by a lesion detection unit configured to detect a lesion from medical image data; and an information presentation unit configured to, in a case where the information representing the operating state is information representing a state in which the detection processing cannot be executed, present information corresponding to a reason why the detection processing cannot be executed.

### BACKGROUND OF THE INVENTION

### Field of the Invention

The present invention relates to an information processing apparatus, an information processing method, and a storage medium.

### Description of the Related Art

There is known CADe (Computer-Aided Detection) in which a computer analyzes a medical image and detects a candidate of a lesion that is an abnormality associated with a disease. In addition, along with the development of AI (Artificial Intelligence) technology, the types of lesions to be covered are increasing.

Japanese Patent Laid-Open No.7-37056 discloses a diagnostic supporting apparatus that divides a medical image into small regions, calculates a feature amount on a small region basis, and displays an image with a display density or display color changing depending on the feature amount superimposed on the medical image. Also, Japanese Patent Laid-Open No. 2005-65944 discloses a diagnostic supporting apparatus that detects an abnormal shadow candidate based on a detection condition, investigates abnormal shadow candidate detection performance for each abnormal shadow detection condition, and displays the investigated detection performance.

However, in a case where a detection result is not displayed by either of the methods of Japanese Patent Laid-Open Nos. 7-37056 and 2005-65944, it is difficult to discriminate whether a lesion cannot be detected even in a case where lesion detection processing is performed or whether no lesion is detected because lesion detection processing is not executed.

The present invention has been made in consideration of the above-described problem, and provides an information processing technique capable of presenting an operating state of lesion detection processing.

### SUMMARY OF THE INVENTION

According to one aspect of the present invention, there is provided an information processing apparatus comprising: an operating state obtaining unit configured to obtain information representing an operating state of detection processing by a lesion detection unit configured to detect a lesion from medical image data; and an information presentation unit configured to, in a case where the information representing the operating state is information representing a state in which the detection processing cannot be executed, present information corresponding to a reason why the detection processing cannot be executed.

Further features of the present invention will become apparent from the following description of exemplary embodiments (with reference to the attached drawings).

### BRIEF DESCRIPTION OF THE DRAWINGS

FIG. 1 is a block diagram showing the configuration of an information processing system according to the first to third embodiments;

FIG. 2 is a block diagram showing the hardware configuration of an information processing apparatus according to the first to third embodiments;

FIG. 3 is a block diagram showing the functional configuration of the information processing apparatus according to the first embodiment;

FIG. 4 is a view showing an example of the user interface screen of the information processing apparatus according to the first embodiment,

FIG. 5 is a flowchart showing processing of the information processing apparatus according to the first embodiment;

FIG. 6 is a block diagram showing the functional configuration of the information processing apparatus according to the second embodiment;

FIG. 7 is a view showing an example of the user interface screen of the information processing apparatus according to the second embodiment;

FIG. 8 is a flowchart showing processing of the information processing apparatus according to the second embodiment;

FIG. 9A is a view showing an example of the user interface screen of the information processing apparatus according to the third embodiment:

FIG. 9B is a view showing an example of the user interface screen of the information processing apparatus according to the third embodiment;

FIGS. 10A and 10B are flowcharts showing processing of the information processing apparatus according to the third embodiment; and

FIG. 11 is a block diagram showing the functional configuration of the information processing apparatus according to the third embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted.

### First Embodiment

In the first embodiment, an information processing apparatus that displays a medical image such as an X-ray CT (Computed Tomography) image or an MRI (Magnetic Resonance Imaging) image will be described.

For example, when a plurality of CADe operate and detect lesion candidates (a “lesion candidate” will be referred to as a “lesion” hereinafter), the information processing apparatus according to this embodiment presents the detection result to a user. The information processing apparatus also decides the type of an associated lesion associated with at least one lesion from the detection result (from a plurality of lesions detected by the plurality of CADe), and presents the result of CADe corresponding to the type of the associated lesion. CADe can cope with a pulmonary nodule, chest wall mass, peritoneal mass, hepatic mass, pancreatic mass, renal mass, colon mass, reticular shadow, honeycomb lung, bronchiectasis, pleuritis, pleural effusion, tenosynovitis, bone erosion, osteitis, pancreatic hypertrophy, pancreatic necrosis, and the like.

### Configuration of Information Processing System

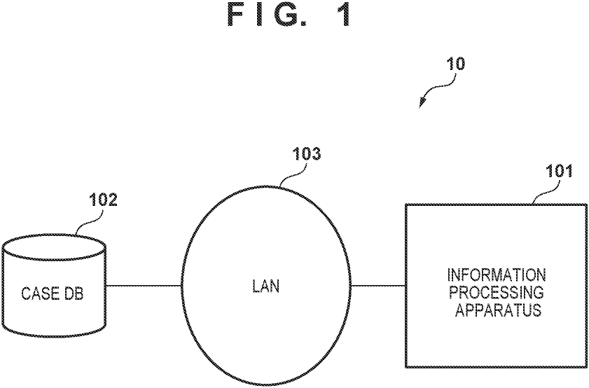

FIG. 1 is a block diagram showing the configuration of an information processing system 10 including the information processing apparatus according to this embodiment. Referring to FIG. 1, the information processing system 10 includes a case database (to be referred to as a case DB hereinafter) 102. an information processing apparatus 101, and a LAN (Local Area Network) 103 (network).

The case DB 102 stores medical image data captured by an apparatus for capturing a medical image, such as a CT apparatus. The case DB 102 also has a database function of providing the medical image data to the information processing apparatus 101 via the LAN 103. More specifically, the case DB 102 according to this embodiment is a known PACS (Picture Archiving and Communication Systems).

### Hardware Configuration

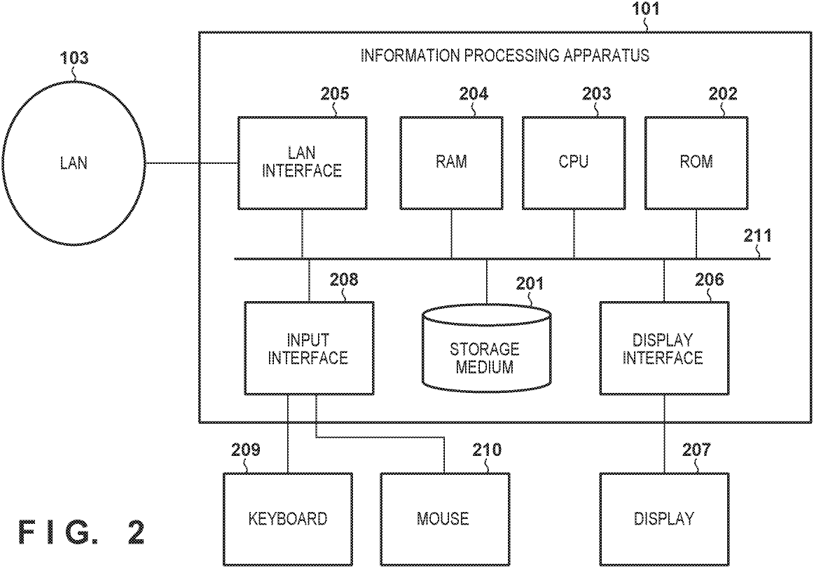

FIG. 2 is a block diagram showing the hardware configuration of the information processing apparatus 101 according to this embodiment. Referring to FIG. 2, the information processing apparatus 101 includes a storage medium 201, a ROM (Read Only Memory) 202, a CPU (Central Processing Unit) 203, and a RAM (Random Access Memory) 204. The information processing apparatus 101 also includes a LAN interface 205, an input interface 208, a display interface 206, and an internal bus 211.

The storage medium 201 is a storage medium such as an HDD (Hard Disk Drive) that stores an OS (Operating System), processing programs configured to perform various kinds of processing according to this embodiment, and various kinds of information. The ROM 202 stores a program configured to initialize hardware and activate the OS, such as a BIOS (Basic Input Output System). The CPU 203 performs arithmetic processing when executing the BIOS, OS, and processing programs. The RAM 204 temporarily stores information when the CPU 203 executes a program. The LAN interface 205 is an interface supporting a standard such as IEEE (Institute of Electrical and Electronics Engineers) 802.3ab and configured to perform communication via the LAN 103. A display 207 (display unit) displays a user interface screen, and the display interface 206 converts screen information to be displayed on the display 207 into a signal and outputs it to the display 207. A keyboard 209 performs key input, a mouse 210 designates a coordinate position on a screen and inputs a button operation, and the input interface 208 receives signals from the keyboard 209 and the mouse 210. The internal bus 211 transfers signals when performing communication between the blocks.

### Functional Configuration

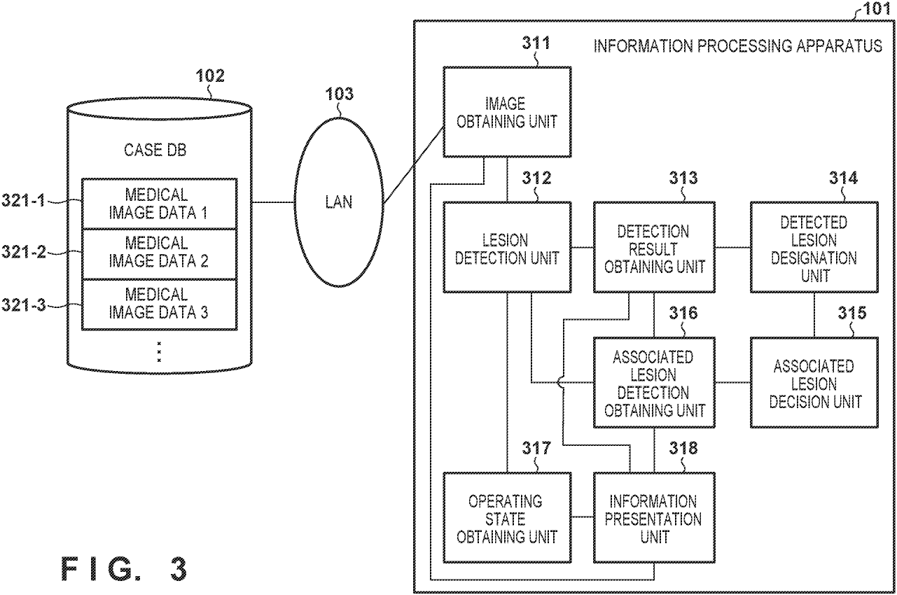

FIG. 3 is a block diagram showing the functional configuration of the information processing apparatus 101 according to this embodiment. Referring to FIG. 3, the information processing apparatus 101 includes an image obtaining unit 311, a lesion detection unit 312, a detection result obtaining unit 313, a detected lesion designation unit 314, an associated lesion decision unit 315, an associated lesion detection obtaining unit 316, an operating state obtaining unit 317, and an information presentation unit 318. These functional configurations are implemented by reading out a predetermined computer program stored in the storage medium 201 to the RAM 204 and executing arithmetic processing by the CPU 203.

In FIG. 3, the case DB 102 stores medical image data 321-i (i = 1, 2, 3,...), and provides the medical image data 321-i (i = 1, 2, 3....) to the information processing apparatus 101 via the LAN 103. The medical image data 321-i (i = 1, 2, 3,...) are, for example. DICOM (Digital Imaging and Communications in Medicine) files.

### Image Obtaining Unit 311

The image obtaining unit 311 obtains the medical image data 321-i (i = 1, 2, 3,...) as an inspection target from the case DB 102 via the LAN interface 205 and the LAN 103. In this embodiment, obtaining of the medical image data 321-i (i = 1, 2, 3,...) complies with DICOM.

### Lesion Detection Unit 312

The lesion detection unit 312 functions as a plurality of CADe and detects a lesion from the obtained medical image data 321-i (i = 1, 2, 3,...). To detect a lesion, a detector that has learned a CNN (Convolutional Neural Network) is used. For the learning of the detector, a set of medical image data and data representing a lesion region in the medical image data is used as supervisors data. The medical image data of the supervisory data is input to the CNN, and the parameters of the CNN are adjusted such that the error between the output value of the CNN and the data representing a lesion region becomes small. The lesion detection unit 312 may be configured to detect one lesion by one CNN or detect a plurality of lesions by one CNN

### Detection Result Obtaining Unit 313

The detection result obtaining unit 313 obtains the detection results of a plurality of lesions by the lesion detection unit 312. The detection results include information representing the type of each detected lesion and information capable of specifying the position of each lesion in the medical image data 321-i (i = 1, 2, 3....). The information representing the type of a lesion is, for example, an ID (identification information) uniquely assigned to each lesion type of the plurality of detected lesions. The information capable of specifying the position of a lesion in the medical image data takes a form of, for example, coordinate information representing the position of a lesion or a mask image capable of displaying the position of a lesion superimposed on the medical image data. The form of the information capable of specifying the position of a lesion may change for each lesion type.

### Detected Lesion Designation Unit 314

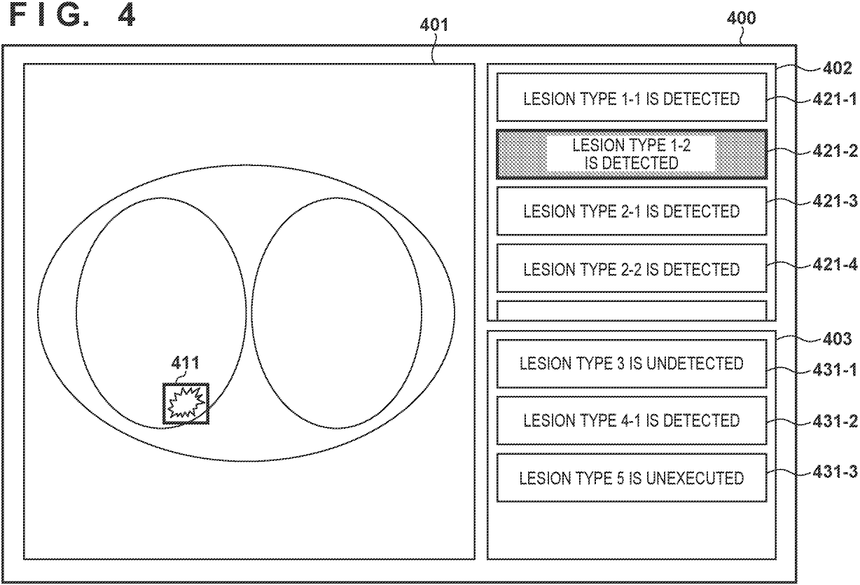

The detected lesion designation unit 314 detects, based on a user operation, an operation of designating at least one lesion (the detection result of a lesion) from the plurality of lesions (the detection results of lesions) obtained by the detection result obtaining unit 313. The user can designate at least one lesion from the detection results of the plurality of lesions by operating the mouse 210 or the keyboard 209. The detection results of the plurality of lesions obtained by the detection result obtaining unit 313 are displayed in a list on a user interface screen 400 (for example, 402 in FIG. 4). The user can change the highlight position of the detection result in a lesion detection result display region 402 using a left click of the mouse 210 or the direction keys or the tab key of the keyboard 209 and designate a lesion (the detection result of a lesion) by operating the enter key or the space key. Based on the operation of the user, the detected lesion designation unit 314 accepts the designation of at least one lesion from the detection results of the plurality of lesions. Note that without using the operation of the user, at least one lesion can be designated from the plurality of lesions (the detection results of lesions) obtained by the detection result obtaining unit 313. For example, in a case where a plurality of lesions are obtained by the detection result obtaining unit 313, the detected lesion designation unit 314 designates a lesion with the largest region or a lesion with the highest severity level.

### Associated Lesion Decision Unit 315

The associated lesion decision unit 315 decides the type of a lesion (associated lesion) associated with the lesion designated by the detected lesion designation unit 314. The associated lesion decision unit 315 decides the type of an associated lesion associated with a detected lesion. As associated lesion type decision processing, for example, the associated lesion decision unit 315 may hold the relationship between lesion types and associated lesion types as information in a table format and decide an associated lesion type based on the table. In a case where a plurality of lesions are designated, the associated lesion decision unit 315 may calculate a logical sum (OR) or a logical product (AND) for each associated lesion type obtained from the table for each designated lesion type and obtain the type of the associated lesion. Which one of OR and AND is to be used may be designated as setting information in advance, selected by the user as needed, or selected in accordance with the combination of designated lesions. Also, the associated lesion decision unit 315 may hold the combination of a plurality of lesion types and an associated lesion type corresponding to that as information in a table format and decide an associated lesion type using the information in the table format. Alternatively, the associated lesion decision unit 315 may decide an associated lesion type from the combination of a plurality of lesion types based on a rule, such as an if-then rule, for associating the combination of lesion types and an associated lesion type.

The table or the rule used to decide an associated lesion type is created based on, for example, medical knowledge. The medical knowledge includes, for example, the relationship between a primary lesion and a metastatic lesion, the relationship of complications, a relationship concerning evaluation of a risk of aggravation, and the like. As for the relationship between a primary lesion and a metastatic lesion, for example, associated lesions of a pulmonary nodule suspected of being a primary lung cancer are masses in a chest wall, peritoneum, liver, pancreas, and the like, which are considered as the metastasis destinations of the primary lung cancer. In addition, for a pulmonary nodule suspected of being a metastatic lung cancer, masses in a colon, kidney, mammary gland, and the like, which are considered as primary lesions, are associated lesions. Also, a reticular shadow in a lung may be a complication of rheumatoid arthritis, and to discriminate it, tenosynovitis, bone erosion, osteitis, and the like are defined as associated lesions. In a case of pancreatitis, to evaluate a risk of aggravation, pancreatic hypertrophy, pancreatic necrosis, and the like are defined as associated lesions.

### Associated Lesion Detection Obtaining Unit 316

The associated lesion detection obtaining unit 316 obtains the detection result of the lesion detection unit 312 for the associated lesion type decided by the associated lesion decision unit 315. That is, the associated lesion detection obtaining unit 316 obtains the detection result of the associated lesion decided by the associated lesion decision unit 315 from the detection results of the plurality of lesions by the lesion detection unit 312.

### Operating State Obtaining Unit 317

The operating state obtaining unit 317 obtains, from the lesion detection unit 312, information (to be also referred to as operating information hereinafter) representing the operating state of detection processing by the lesion detection unit 312 that detects a lesion from medical image data. Also, the operating state obtaining unit 317 obtains information representing the operating state of detection processing by the lesion detection unit 312 that detects an associated lesion in accordance with an associated lesion type. Note that the operating state of detection processing can also be rephrased as the performing state or execution state of detection processing.

The information representing the operating state according to the first embodiment includes information representing, for each lesion type, whether lesion detection (lesion detection processing) by the lesion detection unit 312 is executed (presence/absence of execution). That is, the information representing the operating state includes information representing that lesion detection processing is executed (detected by executing detection processing or undetected even in a case where detection processing is executed) and information representing that lesion detection processing is unexecuted. Also, the information representing the operating state includes information representing that lesion detection processing is being executed, that is, information representing a state in which lesion detection processing is started but not yet completed. In a case where lesion detection processing is being executed, the operating state obtaining unit 317 may obtain, from the lesion detection unit 312, information representing the degree of progress of detection processing and the time remaining until the end of detection processing, and the information presentation unit 318 may present (display) at least one of the degree of progress of detection processing and the time remaining until the end of detection processing, which are obtained by the operating state obtaining unit 317, on the display 207 (display unit) together with the information representing the operating state.

Also, the operating state obtaining unit 317 obtains, from the lesion detection unit 312, information representing the operating state of detection processing for the associated lesion decided by the associated lesion decision unit 315. That is, the operating state obtaining unit 317 obtains information (to be also referred to as associated operating information hereinafter) representing the operating state of detection processing for the associated lesion by the lesion detection unit 312.

The information representing the operating state of detection processing for the associated lesion includes information representing whether lesion detection (associated lesion detection processing) for the associated lesion by the lesion detection unit 312 is executed (presence/absence of execution). In a case of the associated lesion as well, the information representing the operating state of detection processing includes information representing that associated lesion detection processing is executed (detected by executing detection processing or undetected even in a case where detection processing is executed) and information representing that associated lesion detection processing is unexecuted. Also, the information representing the operating state of detection processing for the associated lesion includes information representing that associated lesion detection processing is being executed.

The operating state obtaining unit 317 may obtain information representing the operating state for all lesion types detected by the lesion detection unit 312, or may obtain only information representing the operating state of detection processing for the associated lesion decided by the associated lesion decision unit 315.

### Information Presentation Unit 318

The information presentation unit 318 that presents the information representing the operating state controls display of the user interface screen 400 on the display 207. The information presentation unit 318 displays, on the display 207, an associated lesion detection result obtained by the associated lesion detection obtaining unit 316 and the information representing the operating state obtained by the operating state obtaining unit 317. Also, the information presentation unit 318 displays, on the display 207, the medical image data 321-i (i = 1, 2, 3,...) obtained by the image obtaining unit 311 and a lesion detection result obtained by the detection result obtaining unit 313. When displaying detection results on the display 207, the information presentation unit 318 displays the detection results of associated lesions and the detection results of other lesions (lesions obtained by the detection result obtaining unit 313) such that these can be discriminated.

In this embodiment, the information presentation unit 318 discriminatively displays (presents) the detection results of a plurality of lesions detected by the lesion detection unit 312 and the detection results of associated lesions. An example in which the information presentation unit 318 displays detection results in separate display regions such as a region (for example, 403) where the detection results of associated lesions are displayed and a region (for example, 402) where the detection results of other lesions are displayed, as indicated by the user interface screen 400 in FIG. 4, will be described. However, the present invention is not limited to this example, and the display of detection results by the information presentation unit 318 may be done such that the detection results can be discriminated by, for example, displaying characters or background in different colors or displaying different icon images.

In addition, the information presentation unit 318 displays such that whether it is information representing the operating state of detection processing for an associated lesion can be discriminated. In this embodiment, an example in which display is performed in separate display regions of the display 207, like the detection results, will be described. However, the present invention is not limited to this example, and the information presentation unit 318 may display such that whether it is information representing the operating state of detection processing for an associated lesion can be discriminated by displaying characters or background in different colors or displaying different icon images. In a case where detection processing by the lesion detection unit 312 is executed, and a lesion is detected, the information presentation unit 318 presents the detected lesion.

### User Interface Screen

FIG. 4 is a view showing an example of the user interface screen 400 of the information processing apparatus 101 according to this embodiment. The user interface screen 400 is displayed on the display 207, and various kinds of operations by the user are input via the keyboard 209 or the mouse 210.

In FIG. 4, the user interface screen 400 includes a medical image data display region 401, the lesion detection result display region 402, and the associated lesion detection result display region 403.

The information presentation unit 318 displays, in the medical image data display region 401, medical image data obtained by the image obtaining unit 311. Also, the information presentation unit 318 can perform display control of changing the WL/WW (Window Level/Window Width), the slice position, the magnification ratio, and the like of the image displayed in the medical image data display region 401 in accordance with an operation by the keyboard 209 or the mouse 210.

In addition, the information presentation unit 318 presents (displays) the position of a lesion designated by the detected lesion designation unit 314 on the display of the medical image data. Based on the detection result of the lesion obtained by the detection result obtaining unit 313, the information presentation unit 318 displays an annotation 411 indicating the position of the lesion designated by the detected lesion designation unit 314 on the display of the medical image data in the medical image data display region 401. As a display example of the position of the designated lesion, for example, an image (overlay image) that emphasizes the lesion region by highlight may be displayed on the medical image.

The information presentation unit 318 presents (displays) the detection results of a plurality of lesions and information representing the operating state of detection processing for each lesion together. In the lesion detection result display region 402, the information presentation unit 318 displays detection results (lesion detection results) 421-i (i = 1, 2, 3, 4,...) of lesions obtained by the detection result obtaining unit 313. The lesion detection results 421-i (i = 1, 2. 3, 4,...) correspond to the lesion detection results obtained by the detection result obtaining unit 313. and only the lesion detection results obtained by the detection result obtaining unit 313 are displayed in the lesion detection result display region 402.

In the lesion detection result display region 402, the information presentation unit 318 presents (displays) the lesion detection results 421-i (i = 1, 2, 3, 4,...) and information representing the operating state of detection processing for the lesions obtained by the operating state obtaining unit 317 together. For example, a lesion detection result 421-1 indicates that a lesion of “lesion type 1-1” is detected. Display of “detected” is based on the information representing the operating state, and indicates that the lesion is detected by executing lesion detection (lesion detection processing) by the lesion detection unit 312 (this indicates that detection processing is executed, and the lesion is detected).

In the display of the lesion detection result display region 402, display of “detected” in “lesion type 1-2”, “lesion type 2-1”, and “lesion type 2-2” is the same as described above, and indicates that the lesion types are detected by executing lesion detection processing by the lesion detection unit 312. That is, it indicates that detection processing is executed, and the lesions are detected.

Also, in the lesion detection result display region 402, the detection result of a lesion can be designated by an operation such as a left click of the mouse 210 on the detection result of the lesion. The detected lesion designation unit 314 detects an operation of designating at least one lesion, based on the user operation, from the detection results of the plurality of lesions obtained by the detection result obtaining unit 313.

The information presentation unit 318 displays the designated detection result highlighted such that it can be discriminated from the detection results of other lesions. As an example of highlighting, for example, the frame lines and the background can be highlighted, as indicated by a lesion detection result 421-2. As the highlighting, identification display (for example, characters or an icon image) for discrimination from the detection results of other lesions can be combined with the display of the detection result.

In accordance with the designation of the lesion detection result in the lesion detection result display region 402, the information presentation unit 318 updates the display position of the annotation 411 based on the detection position of the lesion corresponding to the designated lesion detection result.

In addition, based on the associated lesion detection results obtained by the associated lesion detection obtaining unit 316, the information presentation unit 318 updates the display contents in the associated lesion detection result display region 403. For example, in a case where the designation of the lesion detection result in the lesion detection result display region 402 is changed, the information presentation unit 318 updates the display contents in the associated lesion detection result display region 403 based on the detection result of the associated lesion associated with the lesion of the changed designation.

The information presentation unit 318 presents (displays) the detection results of associated lesions and information representing the operating state of detection processing for each associated lesion together. In the associated lesion detection result display region 403, the information presentation unit 318 displays detection results (associated lesion detection results) 431-i (i = 1, 2, 3, 4,....) of associated lesions obtained by the associated lesion detection obtaining unit 316. As The associated lesion detection results 431-i (i = 1, 2, 3, 4,...), the information presentation unit 318 displays the lesion types of the associated lesions.

In the associated lesion detection result display region 403, the information presentation unit 318 displays the associated lesion detection results 431-i (i = 1, 2. 3, 4,...) and information representing the operating state of detection processing for the associated lesions obtained by the operating state obtaining unit 317 together. In the associated lesion detection result display region 403, for example, as indicated by an associated lesion detection result 431-1, even in a case where “lesion type 3” of the associated lesion is undetected, information representing that the associated lesion is undetected is displayed as the presentation of the operating state of detection processing for the associated lesion. Display of “undetected” is based on the information representing the operating state of detection processing for the associated lesion, and indicates that the associated lesion is not detected even in a case where lesion detection (associated lesion detection processing) by the lesion detection unit 312 is executed. That is, this indicates that detection processing is executed, and the lesion (associated lesion) is undetected.

In a case where detection processing for the associated lesion is unexecuted, information representing that the detection processing is unexecuted is displayed as the information representing the operating state. For example, in display of an associated lesion detection result 431-3, concerning the operating state of detection processing for “lesion type 5” of the associated lesion, an indication (“unexecuted”) representing that detection processing is unexecuted is displayed. Display of “unexecuted” is based on the information representing the operating state of detection processing for the associated lesion, and indicates that associated lesion detection processing by the lesion detection unit 312 is unexecuted.

### Processing Procedure

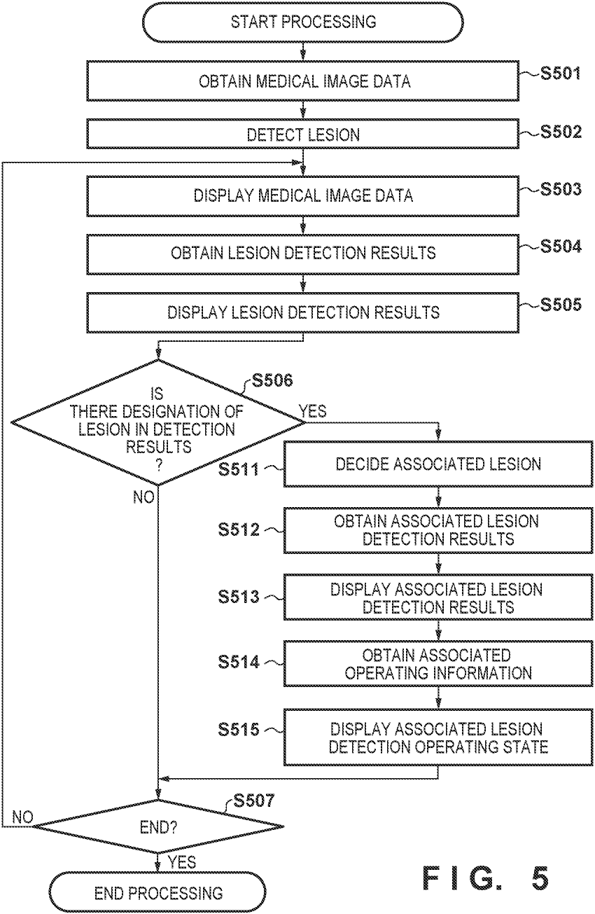

FIG. 5 is a flowchart showing processing of the information processing apparatus 101 according to this embodiment. This processing is started based on an instruction from another system or the user after activation of the information processing apparatus 101 . When starting the processing, a case as the target of the processing is designated.

In step S501, the image obtaining unit 311 obtains the medical image data 321-i (i = 1, 2, 3,...) of the case designated at the time of activation from the case DB 102 via the LAN 103.

In step S502, the lesion detection unit 312 detects lesions from the medical image data 321-i (i = 1, 2, 3,...) obtained in step S501.

In step S503, the information presentation unit 318 displays the medical image data 321-i (i = 1, 2. 3,...) obtained in step S501 in the medical image data display region 401 of the user interface screen 400. The information presentation unit 318 also changes the WL/WW, the slice position, the magnification ratio, and the like of the displayed image based on the operation of the keyboard 209 or the mouse 210.

In step S504, the detection result obtaining unit 313 obtains the detection results of the lesions from the lesion detection unit 312. Each lesion detection result includes information representing the type of detected lesion and information capable of specifying the position of the lesion in the medical image data 321-i (i = 1, 2, 3,...).

In step S505, the information presentation unit 318 displays the lesion detection results 421-i (i = 1, 2, 3, 4,...) in the lesion detection result display region 402 of the user interface screen 400 based on the lesion detection results obtained in step S504.

In step S506, the detected lesion designation unit 314 determines the presence/absence of an operation of designating a lesion from the plurality of lesion detection results displayed in the lesion detection result display region 402. That is, the detected lesion designation unit 314 detects the presence/absence of the operation of designating a detected lesion based on an input from the keyboard 209 or the mouse 210. In a case where the detected lesion designation unit 314 detects a designation of a lesion in step S506 (YES in step S506), the process advances to step S511. On the other hand, in a case where a designation of a lesion is not detected in the determination of step S506 (NO in step S506), the process advances to step S507.

In step S507, the OS (Operating System) (not shown) determines whether to end the processing of the information processing apparatus 101. The end of the processing is determined based on the presence/absence of an ending operation such as an OS shutdown operation, a power-off operation, an operation of closing a window, or a process stop. In a case where the OS detects the ending operation (YES in step S507), the processing is ended. In a case where the ending operation is not detected (NO in step S507), the process returns to step S503, and the same processing as described above is repeated from step S503.

On the other hand, in a case where the detected lesion designation unit 314 detects a designation of a lesion (YES in step S506), in step S511, the associated lesion decision unit 315 decides an associated lesion associated with the designated lesion based on the lesion detection result designation detected in step S506.

In step S512, the associated lesion detection obtaining unit 316 obtains a detection result for the associated lesion decided in step S511.

In step S513, the information presentation unit 318 displays the associated lesion detection results 431-i (i = 1, 2, 3,...) in the associated lesion detection result display region 403 of the user interface screen 400 based on the associated lesion detection result obtained in step S512.

In step S514, the operating state obtaining unit 317 obtains information (associated operating information) representing the operating state of detection processing for the associated lesion decided in step S511. As the information representing the operating state, the operating state obtaining unit 317 obtains information representing whether lesion detection (associated lesion detection processing) for the associated lesion by the lesion detection unit 312 is executed (presence/absence of execution). The information representing the operating state of detection processing for the associated lesion includes information representing that associated lesion detection processing is executed (detected by executing detection processing or undetected even in a case where detection processing is executed), information representing that associated lesion detection processing is unexecuted, and information representing that associated lesion detection processing is being executed.

In step S515, the information presentation unit 318 displays (presents) the information representing the operating state of detection processing for the associated lesion, which is obtained in step S514. as the associated lesion detection result 431-i (i = 1, 2, 3,...) in the associated lesion detection result display region 403. In a case where the processing of step S515 is ended, the process advances to step S507.

In step S507, in a case where the OS detects the ending operation (YES in step S507), the processing is ended. In a case where the ending operation is not detected (NO in step S507), the process returns to step S503, and the same processing as described above is repeated from step S503.

According to this embodiment, it is possible to present the operating state of detection processing for detecting a lesion. Also, according to this embodiment, in a case where the user designates a detected lesion in the display on the user interface screen, the type of an associated lesion associated with the designated lesion is automatically decided, and the detection result of the associated lesion is displayed. Hence, even in a case where the number of lesions as the detection target increases, the presence/absence of the detection result of another associated lesion can easily be found.

Also, since the operating state of lesion detection processing is displayed, even in a case where no lesion is detected, it is possible to easily discriminate whether no lesion is detected even in a case where lesion detection processing is executed, or no lesion is detected because lesion detection processing is not executed.

### Modification of First Embodiment

The information processing apparatus 101 may be, for example, an image processing workstation, an electronic medical chart, an integration viewer configured to integrally display information from a plurality of types of apparatuses, or an apparatus for capturing a medical image, such as an ultrasonic diagnostic apparatus.

The lesion detection unit 312 may be located on another apparatus such as an image processing server connected to the information processing apparatus 101 via a network. Also, the lesion detection unit 312 may detect a lesion at the timing of capturing of the medical image data 321-i (i = 1, 2, 3,...), or may detect a lesion at the timing of storing the medical image data 321-i (i = 1, 2, 3,...) in the case DB 102. In addition, when capturing medical image data or storing medical image data in the case DB 102, the lesion detection unit 312 may detect a lesion by background processing and store the detection result in a storage device such as the case DB 102. In this case, the detection result obtaining unit 313 obtains the detection result from the storage device.

The lesion detection unit 312 may detect a plurality of types of lesions from the obtained medical image data 321-i (i = 1, 2, 3,...) using a method other than the CNN, such as SVM (Support Vector Machine). Also, the associated lesion decision unit 315 may extract medical knowledge by language processing of a past interpretation report, a paper, or a diagnostic guideline and create a table or a rule used to decide an associated lesion.

### Second Embodiment

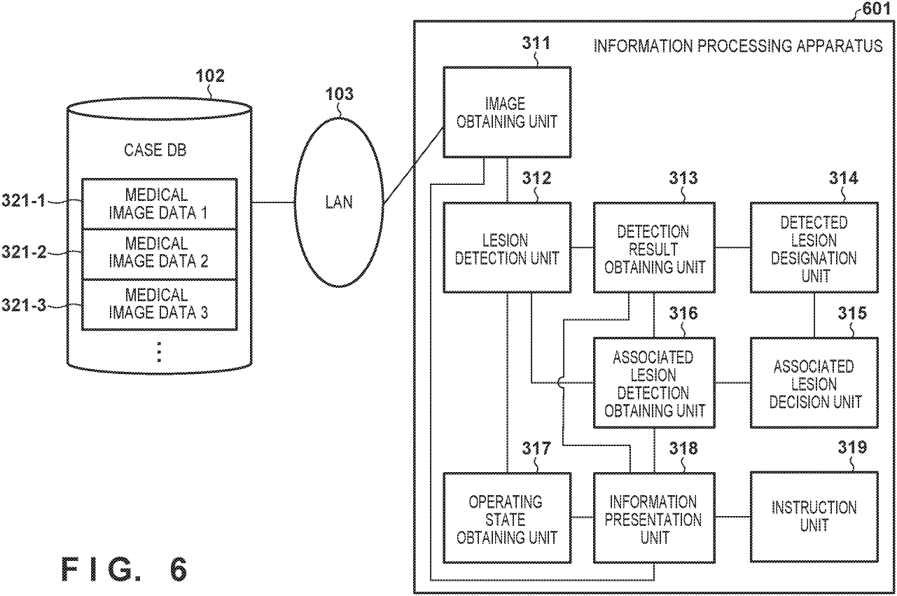

In a case where associated lesion detection processing is unexecuted in information representing the operating state of detection processing for an associated lesion, an information processing apparatus 601 according to the second embodiment presents information representing whether the unexecuted detection processing can be executed or not, in addition to the information processing apparatus 101 of the first embodiment. In a case where associated lesion detection processing is unexecuted, and the unexecuted detection processing can be executed, an instruction unit 319 (FIG. 6) of the information processing apparatus 601 instructs a lesion detection unit 312 to execute the unexecuted detection processing. Note that the system configuration of the information processing apparatus 601 according to the second embodiment is the same as in FIG. 1, and the hardware configuration is the same as in the first embodiment described with reference to FIG. 2. Hence, a description of these will be omitted.

In the processing of the information processing apparatus 601 to be described in the second embodiment, information representing the operating state includes information representing whether lesion detection (lesion detection processing) by the lesion detection unit 312 is executed (presence/absence of execution), and information representing whether lesion detection processing can be executed or not. That is, the information representing the operating state includes information representing that associated lesion detection processing is executed (detected by executing detection processing or undetected even in a case where detection processing is executed), information representing that lesion detection processing is unexecuted, and information representing that lesion detection processing is being executed. Note that in this embodiment, the unexecuted detection processing will be described using an associated lesion displayed in an associated lesion detection result display region 403 as an example. This also applies to a case where detection processing for a lesion displayed in a lesion detection result display region 402 is unexecuted.

### Functional Blocks

FIG. 6 is a block diagram showing the functional configuration of the information processing apparatus 601 according to this embodiment. The same reference numerals as the functional blocks of the information processing apparatus 101 according to the first embodiment described with reference to FIG. 3 denote the same functional blocks, and a description thereof will be omitted. In FIG. 6, the functional configuration of the information processing apparatus 601 is different in that the instruction unit 319 is provided, in addition to the information processing apparatus 101 according to the first embodiment. The functional configuration of the instruction unit 319 is implemented by reading out a predetermined computer program stored in a storage medium 201 to a RAM 204 and executing arithmetic processing by a CPU 203.

### Instruction Unit 319

In a case where detection processing is unexecuted, and the unexecuted detection processing can be executed, in the information representing the operating state of detection processing, the instruction unit 319 instructs the lesion detection unit 312 to execute the detection processing. In this embodiment, a configuration in which the instruction of unexecuted associated lesion detection processing by the instruction unit 319 is executed upon receiving a user confirmation via an instruction confirmation window 404 will be described.

### User Interface Screen

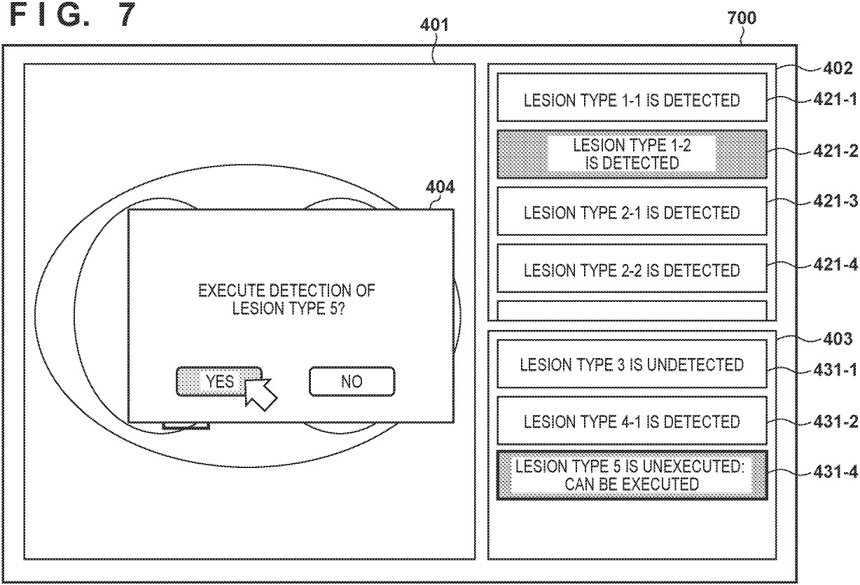

FIG. 7 is a view showing an example of a user interface screen 700 of the information processing apparatus 601 according to this embodiment. Note that the same reference numerals as in the user interface screen 400 according to the first embodiment described with reference to FIG. 4 denote the same parts, and a description thereof will be omitted.

An information presentation unit 318 controls display of the user interface screen 700 on a display 207. The user interface screen 700 according to this embodiment has the same screen configuration as the user interface screen 400 described in the first embodiment. In this embodiment, additionally, in a case where detection processing of a lesion (associated lesion) is unexecuted, the information presentation unit 318 presents, on the user interface screen 700, information representing whether unexecuted detection processing can be executed or not in associated lesion detection results 431-i (i = 1, 2, 3....).

As shown in FIG. 7, the information presentation unit 318 displays an indication (“unexecuted”) representing that detection processing of “lesion type 5” of an associated lesion is unexecuted and an indication (“executable”) representing that the detection processing can be executed together with the display of an associated lesion detection result 431-4.

In a case where the user designates the display of the associated lesion detection result 431-4, the information presentation unit 318 displays the frame lines and the background highlighted such that the designated display of the associated lesion detection result 431-4 can easily discriminated from the detection results (for example, 431-1 and 431-2) of other associated lesions. Note that as the highlighting, identification display (for example, characters or an icon image) can be combined with the display of the detection result to make discrimination from the detection results of other associated lesions.

In a case where the user designates the display of the associated lesion detection result 431-4, the information presentation unit 318 displays, on the display 207, the instruction confirmation window 404 for requesting confirmation of the user concerning whether to execute the unexecuted detection processing. That is, in a case where the associated lesion detection result (for example, 431-4) for which detection processing is unexecuted, and the unexecuted detection processing can be executed is designated by the designation operation of the user, the information presentation unit 318 displays the instruction confirmation window 404 on the display 207.

In the instruction confirmation window 404, the user can instruct, by operating a keyboard 209 or a mouse 210, whether to execute the unexecuted detection processing. In a case where the user instructs “YES” in the instruction confirmation window 404, the instruction unit 319 instructs the lesion detection unit 312 to execute detection processing of the associated lesion. On the other hand, in a case where the user instructs “NO” in the instruction confirmation window 404, the instruction unit 319 does not instruct the lesion detection unit 312 to execute detection processing of the associated lesion.

### Processing Procedure

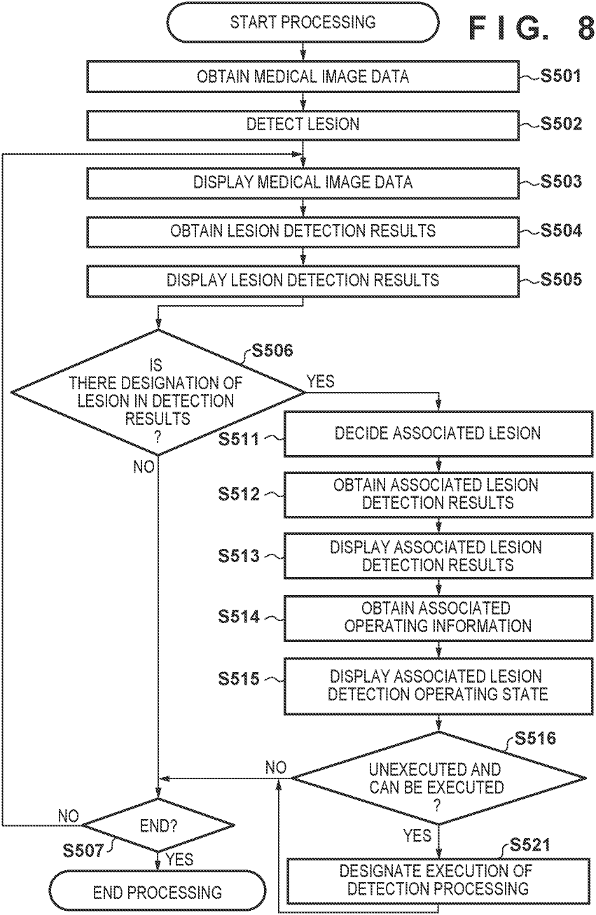

FIG. 8 is a flowchart showing processing of the information processing apparatus 601 according to this embodiment. Note that the same step numbers as the steps of the processing procedure of the first embodiment described with reference to FIG. 5 denote the same steps, and a description thereof will be omitted.

In step S516, in a case where the designation operation of the user is performed on the display (step S515) of the associated lesion detection results 431-i (i = 1, 2, 3,...), the instruction unit 319 determines, based on the information representing the operating state of detection processing, whether detection processing is unexecuted, and the unexecuted detection processing can be executed or not. In a case where detection processing is unexecuted, and the unexecuted detection processing cannot be executed in the determination processing of step S516 (NO in step S516), the instruction unit 319 returns the process to step S507. On the other hand, in a case where detection processing is unexecuted, and the unexecuted detection processing can be executed (YES in step S516), the instruction unit 319 advances the process to step S521.

In step S521, the instruction unit 319 instructs the lesion detection unit 312 to execute detection processing of the associated lesion for which it has been determined in step S516 that the information representing the operating state is “unexecuted”, and the detection processing can be executed. Then, the process returns to step S507 to determine whether to end the processing.

In this embodiment, the information presentation unit 318 displays, on the display 207. the instruction confirmation window 404 as shown in FIG. 7 to request confirmation of the user concerning whether to execute the detection processing that is unexecuted and can be executed. In a case where the user instructs “YES” in the instruction confirmation window 404, the instruction unit 319 instructs the lesion detection unit 312 to execute detection processing of the associated lesion. In a case where the user instructs “NO” in the instruction confirmation window 404, the instruction unit 319 does not instruct the lesion detection unit 312 to execute detection processing of the associated lesion but ends the processing of this step and returns the process to step S507.

According to this embodiment, it is possible to present the operating state of detection processing for detecting a lesion. Also, according to this embodiment, in a case where the user designates a detected lesion in the display on the user interface screen, the type of an associated lesion associated with the designated lesion is automatically decided, and the detection result of the associated lesion is displayed. Hence, even in a case where the number of lesions as the detection target increases, the presence/absence of the detection result of another associated lesion can easily be found.

Also, since the operating state of lesion detection processing is displayed, even in a case where no lesion is detected, it is possible to easily discriminate whether no lesion is detected even in a case where lesion detection processing is executed, or no lesion is detected because lesion detection processing is not executed.

Furthermore, in a case where lesion detection processing is unexecuted and can be executed, lesion detection processing is instructed, thereby easily executing the instructed unexecuted lesion (associated lesion) detection processing.

### Modification of Second Embodiment

In the second embodiment, the instruction of unexecuted associated lesion detection processing by the instruction unit 319 is executed upon receiving a user confirmation via the instruction confirmation window 404. However, the instruction unit 319 may instruct execution of detection processing by the lesion detection unit 312 based on the information representing the operating state. That is, without receiving the user confirmation, in a case where detection processing is unexecuted, and the unexecuted detection processing can be executed based on the information representing the operating state, the instruction unit 319 may instruct the lesion detection unit 312 to execute the unexecuted lesion detection processing.

### Third Embodiment

The configuration of an information processing system 10 including an information processing apparatus 1101 according to this embodiment is the same as in FIG. 1, and the hardware configuration of the information processing apparatus 1101 is the same as the hardware configuration of the information processing apparatus 101 according to the first embodiment described with reference to FIG. 2.

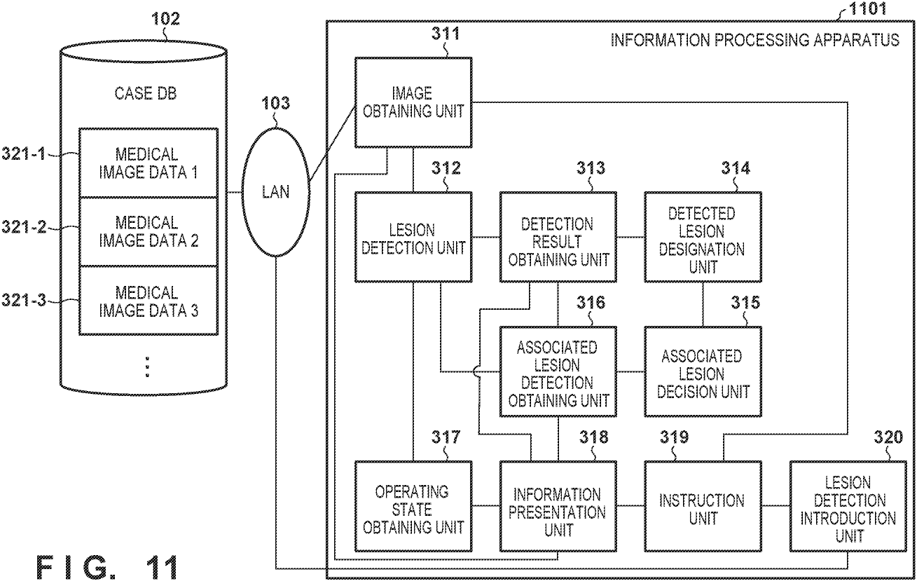

FIG. 11 is a block diagram showing the functional configuration of the information processing apparatus 1101 according to this embodiment. The same reference numerals as the functional blocks of the information processing apparatus 101 according to the first embodiment and the functional blocks of the information processing apparatus 601 according to the second embodiment denote the same functional blocks, and a description thereof will be omitted. In FIG. 11, the functional configuration of the information processing apparatus 1101 is different in that a lesion detection introduction unit 320 is provided, in addition to the information processing apparatus 101 according to the first embodiment and the information processing apparatus 601 according to the second embodiment. The functional configuration of the lesion detection introduction unit 320 is implemented by reading out a predetermined computer program stored in a storage medium 201 to a RAM 204 and executing arithmetic processing by a CPU 203.

In the processing of the information processing apparatus 1101 to be described in the third embodiment, information representing the operating state includes information representing whether lesion detection (lesion detection processing) by a lesion detection unit 312 is executed (presence/absence of execution), information representing whether lesion detection processing can be executed or not, and information corresponding to the reason why the detection processing cannot be executed (the reason for “inexecutable”) in a case where the unexecuted detection processing cannot be executed (in a case of “inexecutable”). In a case where the information representing the operating state is information representing a state in which the detection processing cannot be executed, an information presentation unit 318 presents the information corresponding to the reason why the detection processing cannot be executed.

In a case where unexecuted detection processing cannot be executed (in a case of “inexecutable”) in the information representing the operating state, the information presentation unit 318 of the information processing apparatus 1101 according to this embodiment presents information corresponding to the reason why the detection processing cannot be executed (the reason for “inexecutable”), in addition to the configuration of the information processing apparatus 601 of the second embodiment. Note that in this embodiment, the “inexecutable” detection processing will be described using an associated lesion displayed in an associated lesion detection result display region 403 as an example. This also applies to a case where detection processing for a lesion displayed in a lesion detection result display region 402 is inexecutable.

### User Interface Screen

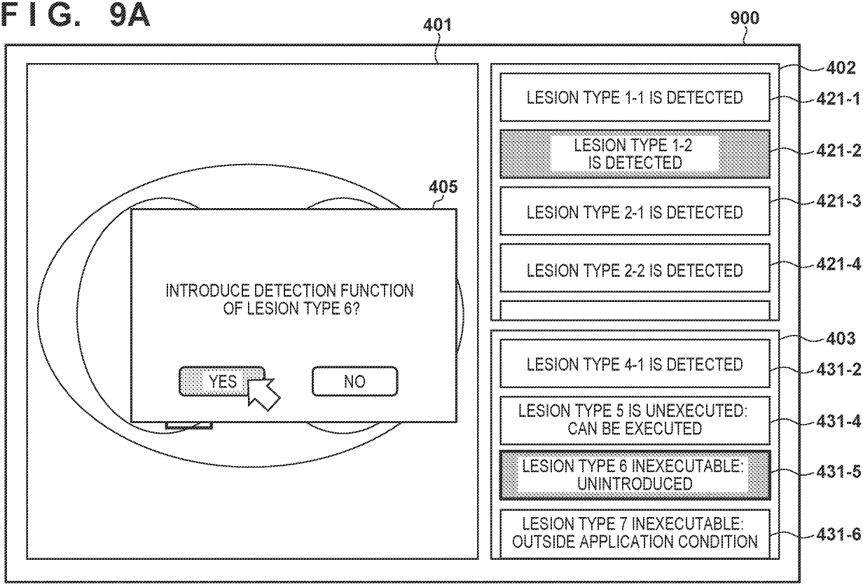

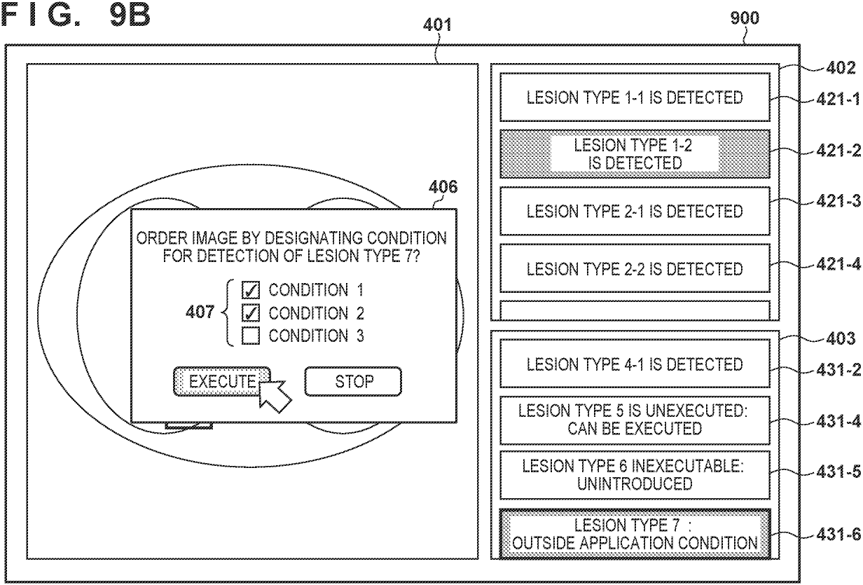

FIGS. 9A and 9B are views showing an example of a user interface screen 900 of the information processing apparatus 1101 according to this embodiment. Note that the same reference numerals as in the user interface screens according to the first embodiment described with reference to FIG. 4 and the second embodiment described with reference to FIG. 7 denote the same parts, and a description thereof will be omitted.

The information presentation unit 318 controls display of the user interface screen 900 on a display 207. The user interface screen 900 (FIGS. 9A and 9B) according to this embodiment has the same screen configuration as the user interface screen 700 described in the second embodiment.

In this embodiment, in a case where unexecuted detection processing is inexecutable in the information representing the operating state, the information presentation unit 318 presents, on the user interface screen 900, information corresponding to the reason for “inexecutable”.

In this embodiment, the information corresponding to the reason why the detection processing cannot be executed includes, concerning the designated lesion (associated lesion), information representing “unintroduced” in which a lesion detection function is not introduced to the lesion detection unit 312, and information representing that the medical image data is data outside an execution condition of detection processing by the lesion detection unit 312 based on comparison between attributes the image data and the lesion detection unit 312.

In the display examples shown in FIGS. 9A and 9B, the information presentation unit 318 displays an indication (“inexecutable”) representing that detection processing of “lesion type 6” of the associated lesion cannot be executed, and an indication (“unintroduced”) representing that the detection function for the designated lesion (associated lesion) is not introduced to the lesion detection unit 312 together with the display of an associated lesion detection result 431-5.

Also, the information presentation unit 318 displays an indication (“inexecutable”) representing that detection processing of “lesion type 7” of the associated lesion cannot be executed, and an indication (“outside execution condition”) representing that the medical image data is data that is not suitable for detection processing and is outside the execution condition together with the display of an associated lesion detection result 431-6.

As the screen configuration, the user interface screen 900 includes an introduction confirmation window 405 (for example, FIG. 9A) configured to confirm whether to introduce the detection function, and an image obtaining confirmation window 406 (for example, FIG. 9B) configured to confirm whether to obtain image data (medical image data) suitable for the execution condition. In accordance with the reason why detection processing is inexecutable (“unintroduced” or “outside application condition”), the information presentation unit 318 presents one of the introduction confirmation window 405 and the image obtaining confirmation window 406 on the user interface screen 900.

### Introduction Confirmation Window 405: Fig. 9A

FIG. 9A is a view showing the user interface screen 900 on which the introduction confirmation window 405 is displayed. As shown in FIG. 9A, in a case where the user designates display of the associated lesion detection result 431-5 (“inexecutable”. “unintroduced”), the information presentation unit 318 displays the associated lesion detection result 431-5 while highlighting the frame lines and the background such that it can easily be discriminated from the display of the detection results (for example, 431-2, 431-4, and 431-6) of other associated lesions. Note that as the highlighting, identification display (for example, characters or an icon image) can be combined with the display of the detection result to make discrimination from the detection results of other associated lesions.

In a case where the user designates the display of the associated lesion detection result 431-5, the information presentation unit 318 displays, on the display 207, the introduction confirmation window 405 for requesting confirmation of the user concerning whether to introduce the unintroduced detection function to the lesion detection unit 312. That is, in a case where display of the associated lesion detection result (for example, 431-5) for which detection processing is inexecutable, and the detection function corresponding to the unexecuted detection processing is unintroduced to the lesion detection unit 312 is designated by the designation operation of the user, the information presentation unit 318 displays the introduction confirmation window 405 on the display 207.

In the introduction confirmation window 405, the user can instruct, by operating a keyboard 209 or a mouse 210, whether to introduce the unintroduced detection function to the lesion detection unit 312. Here, introduction of the lesion detection function can include installing lesion detection software to be executed by the lesion detection unit 312 to detect a lesion and inputting information (for example, authentication information such as an activation key) for activating the unintroduced lesion detection function in the lesion detection software configured to detect a lesion.

In a case where the user instructs “NO” in the introduction confirmation window 405, an instruction unit 319 does not perform processing associated with introduction of the unintroduced lesion detection function. On the other hand, in a case where the user instructs “YES” in the introduction confirmation window 405, the instruction unit 319 executes the following processing.

### Installation of Lesion Detection Software

In this embodiment, in a case where the information corresponding to the reason why detection processing cannot be executed is information representing “unintroduced”, the instruction unit 319 instructs the lesion detection introduction unit 320 to introduce the unintroduced lesion detection function. In a case where the user instructs “YES” in the introduction confirmation window 405, the instruction unit 319 instructs the lesion detection introduction unit 320 to introduce the unintroduced lesion detection function. The lesion detection introduction unit 320 downloads the lesion detection software configured to implement the unintroduced lesion detection function from an external server (not shown) to a storage medium 201 via a LAN interface 205 and a LAN 103 and stores the lesion detection software. The lesion detection introduction unit 320 registers the lesion detection software in the lesion detection unit 312. The lesion detection unit 312 reads out the registered lesion detection software and executes it, thereby executing the inexecutable lesion detection processing.

### Input of Activation Key

In a case where the lesion detection software configured to implement the unintroduced lesion detection function is stored in the storage medium 201 in advance, in a case where the user instructs “YES” in the introduction confirmation window 405, the instruction unit 319 instructs the lesion detection introduction unit 320 to introduce the unintroduced lesion detection function.

The lesion detection introduction unit 320 downloads an activation key (for example, a predetermined character string) used to activate the unintroduced lesion detection function in the lesion detection software from an external server (not shown) via the LAN interface 205 and the LAN 103 and temporarily stores the activation key in the RAM 204 or the like. The lesion detection introduction unit 320 registers the activation key in the lesion detection unit 312. The lesion detection unit 312 reads out the lesion detection software from the storage medium 201 and sets the registered key, thereby executing the inexecutable lesion detection processing.

### Image Obtaining Confirmation Window 406: Fig. 9B

FIG. 9B is a view showing the user interface screen 900 on which the image obtaining confirmation window 406 is displayed. As shown in FIG. 9B, in a case where the user designates display of the associated lesion detection result 431-6 (“inexecutable”, “outside application condition”), the information presentation unit 318 displays the associated lesion detection result 431-6 while highlighting the frame lines and the background such that it can easily be discriminated from the display of the detection results (for example. 431-2, 431-3, and 431-5) of other associated lesions. Note that as the highlighting, identification display (for example, characters or an icon image) can be combined with the display of the detection result to make discrimination from the detection results of other associated lesions.

In a case where the user designates the display of the associated lesion detection result 431-6, the information presentation unit 318 displays, on the display 207, the image obtaining confirmation window 406 for requesting confirmation of the user concerning whether to obtain image data (medical image data) suitable for the execution condition. That is, in a case where display of the associated lesion detection result (for example, 431-6) for which detection processing is inexecutable, and the medical image data is data outside the execution condition (outside application condition) is designated by the designation operation of the user, the information presentation unit 318 displays the image obtaining confirmation window 406 on the display 207.

The information presentation unit 318 presents the image obtaining confirmation window 406 including a condition designation portion 407 (checkbox) capable of adding or changing a condition to obtain medical image data that satisfies the execution condition of detection processing. The user can add or change the image obtaining condition by designating the condition designation portion 407 by operating the keyboard 209 or the mouse 210. In the image obtaining confirmation window 406 shown in FIG. 9B, a state in which condition 1 and condition 2 are designated (with check marks), and condition 3 is excluded from the image obtaining condition (without a check mark) is displayed by the designation of the condition designation portion 407. Here, the condition for image obtaining includes various conditions, and can include, for example, a modality for capturing a medical image, a reconstruction function, a contrast condition, a time phase, an image capturing range, and the like.

In the image obtaining confirmation window 406, the user can instruct an image obtaining unit 311, by operating the keyboard 209 or the mouse 210, whether to obtain image data (medical image data) suitable for the execution condition. In a case where the user instructs “stop” in the image obtaining confirmation window 406, the instruction unit 319 does not instruct the image obtaining unit 311 to obtain image data (medical image data) suitable for the execution condition. On the other hand, in a case where the user instructs “execute” in the image obtaining confirmation window 406. the instruction unit 319 instructs the image obtaining unit 311 to obtain image data (medical image data) suitable for the designated execution condition. That is, in this embodiment, in a case where the information corresponding to the reason why detection processing cannot be executed is information representing that the data is outside the execution condition, the instruction unit 319 instructs the image obtaining unit 311 to obtain medical image data that satisfies the execution condition of detection processing.

The image obtaining unit 311 may generate the medical image data satisfying the execution condition from already obtained medical image data, or may transmit an order for obtaining to an ordering system or the like to obtain the medical image data satisfying the execution condition from the outside.

Upon receiving the image obtaining instruction from the instruction unit 319, the image obtaining unit 311 performs obtaining processing of image data (medical image data) suitable for the designated condition. When obtaining the medical image data from the outside, the image obtaining unit 311 outputs, via the LAN 103, an image capturing instruction including a condition of image reconstruction and the like to an ordering system including an HIS (Hospital Information Systems) or an RIS (Radiology Information Systems). The image obtaining unit 311 may obtain image data captured by the modality of the HIS or RIS based on the designated condition, or may obtain image data suitable for the condition from the case DB 102 or a PACS (Picture Archiving and Communication Systems) (not shown). The image obtaining unit 311 stores the obtained image data in the storage medium 201. The image data obtained by the image obtaining unit 311 is suitable for the execution condition (application condition) of lesion detection, and the lesion detection unit 312 can execute inexecutable lesion detection processing by using the image data obtained by the image obtaining unit 311.

### Processing Procedure

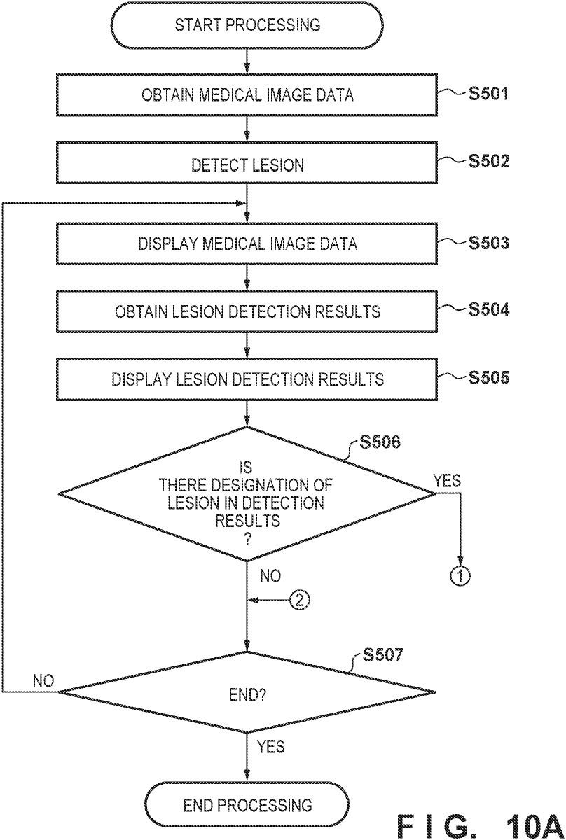

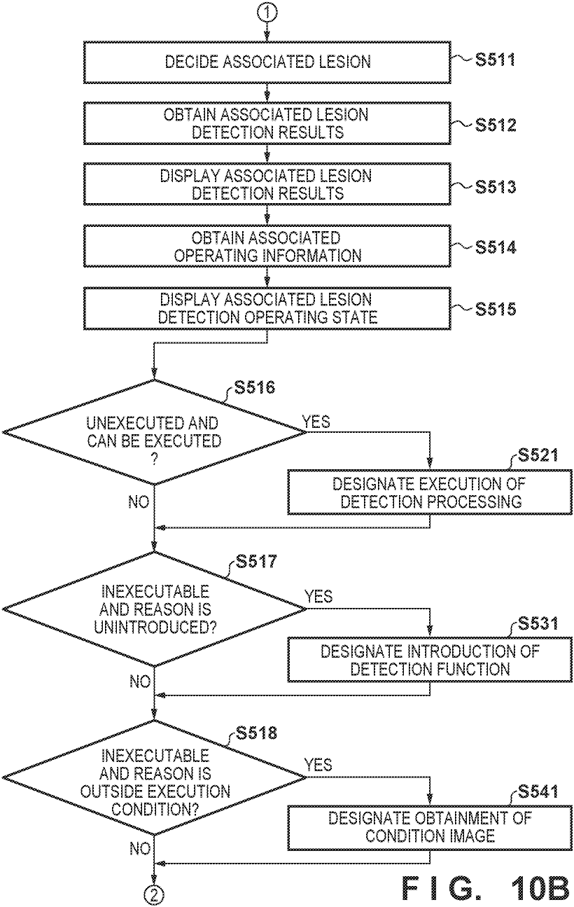

FIGS. 10A and 10B are flowcharts showing processing of the information processing apparatus 1101 according to this embodiment. Note that the same step numbers as the steps of the processing procedure of the first embodiment described with reference to FIG. 5 and the steps of the processing procedure of the second embodiment described with reference to FIG. 8 denote the same steps, and a description thereof will be omitted.

In step S517, in a case where the designation operation of the user in the display (step S515) of the associated lesion detection results 431-i (i = 1, 2, 3,...) is performed, the instruction unit 319 determines, based on the information representing the operating state of detection processing, whether detection processing is inexecutable, and the reason for “inexecutable” is “unintroduced”. In a case where detection processing is inexecutable, and the reason for “inexecutable” is “unintroduced” in the determination processing of step S517 (YES in step S517), the instruction unit 319 advances the process to step S531.

In step S531, the instruction unit 319 instructs the lesion detection introduction unit 320 to introduce the unintroduced lesion detection function. Upon receiving the instruction from the instruction unit 319, the lesion detection introduction unit 320 performs introduction processing for introducing the lesion detection function. Detailed processing for introducing the lesion detection function has been described above with reference to the introduction confirmation window 405.

In this embodiment, when executing the instruction, the user can instruct, in the introduction confirmation window 405, whether to introduce the unintroduced detection function to the lesion detection unit 312 by operating the keyboard 209 or the mouse 210. In a case where the user instructs “YES” in the introduction confirmation window 405, the instruction unit 319 instructs the lesion detection introduction unit 320 to introduce the unintroduced lesion detection function. In a case where the user instructs “NO” in the introduction confirmation window 405, the instruction unit 319 does not perform processing concerning introduction of the unintroduced lesion detection function but ends the processing of this step and advances the process to step S518.

On the other hand, in a case where detection processing is inexecutable, and the reason for “inexecutable” is not “unintroduced” in the determination processing of step S517 (NO in step S517), the instruction unit 319 advances the process to step S518.

In step S518, in a case where the designation operation of the user in the display (step S515) of the associated lesion detection results 431-i (i = 1, 2, 3,...) is performed, the instruction unit 319 determines, based on the information representing the operating state of detection processing, whether detection processing is inexecutable, and the reason for “inexecutable” is “outside execution condition”. In a case where detection processing is inexecutable, and the reason for “inexecutable” is “outside execution condition” in the determination processing of step S518 (YES in step S518), the instruction unit 319 advances the process to step S541.

In step S541, the instruction unit 319 instructs the image obtaining unit 311 to obtain image data (medical image data) suitable for the designated condition. Upon receiving the image data (medical image data) obtaining instruction from the instruction unit 319, the image obtaining unit 311 performs obtaining processing for obtaining image data (medical image data) suitable for the designated condition. Detailed processing has been described above with reference to the image obtaining confirmation window 406.

In this embodiment, when executing the instruction, the user can instruct the image obtaining unit 311, in the image obtaining confirmation window 406. whether to obtain image data (medical image data) suitable for the execution condition by operating the keyboard 209 or the mouse 210. In a case where the user instructs “execute” in the image obtaining confirmation window 406, the instruction unit 319 instructs the image obtaining unit 311 to obtain image data (medical image data) suitable for the designated condition. In a case where the user instructs “stop” in the image obtaining confirmation window 406. the instruction unit 319 does not instruct the image obtaining unit 311 to obtain image data (medical image data) suitable for the execution condition but ends the processing of this step.

On the other hand, in a case where detection processing is inexecutable, and the reason for “inexecutable” is not “outside execution condition” in the determination processing of step S518 (NO in step S518), the instruction unit 319 returns the process to step S507.

According to this embodiment, it is possible to present the operating state of detection processing for detecting a lesion. Also, according to this embodiment, in a case where the user designates a detected lesion in the display on the user interface screen, the type of an associated lesion associated with the designated lesion is automatically decided, and the detection result of the associated lesion is displayed. Hence, even in a case where the number of lesions as the detection target increases, the presence/absence of the detection result of another associated lesion can easily be found.

Since the operating state of lesion detection processing is displayed, even in a case where no lesion is detected, it is possible to easily discriminate whether no lesion is detected even in a case where lesion detection processing is executed, or no lesion is detected because lesion detection processing is not executed.

In a case where lesion detection processing is unexecuted and can be executed, lesion detection processing is instructed, thereby easily executing the instructed unexecuted lesion (associated lesion) detection processing.

Even in a case where detection processing is inexecutable, and the detection function corresponding to unexecuted detection processing is unintroduced, introduction of the lesion detection function is instructed, thereby easily introducing the necessary lesion detection function to the lesion detection unit 312.

Even in a case where detection processing is inexecutable, and the medical image data is data outside the execution condition, obtaining of medical image data satisfying the execution condition is instructed, thereby easily obtaining medical image data necessary for executing lesion detection processing.

According to the present invention, it is possible to present the operating state of lesion detection processing.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2021-184222, filed Nov. 11, 2021. which is hereby incorporated by reference herein in its entirety.

What is claimed is:

1. An information processing apparatus comprising:
an operating state obtaining unit configured to obtain information representing an operating state of detection processing by a lesion detection unit configured to detect a lesion from medical image data; and
an information presentation unit configured to, in a case where the information representing the operating state is information representing a state in which the detection processing cannot be executed, present information corresponding to a reason why the detection processing cannot be executed.

2. The apparatus according to claim 1, wherein in a case where the detection processing by the lesion detection unit is executed, and a lesion is detected, the information presentation unit presents the detected lesion.

3. The apparatus according to claim 1, further comprising:
an image obtaining unit configured to obtain the medical image data;
the lesion detection unit configured to detect a lesion from the medical image data;
an associated lesion decision unit configured to decide a type of an associated lesion associated with the lesion; and
an associated lesion detection obtaining unit configured to obtain a detection result of the associated lesion by the lesion detection unit in accordance with the type of the associated lesion,
wherein the operating state obtaining unit obtains information representing an operating state of detection processing for the associated lesion by the lesion detection unit, and
the information presentation unit further presents the information representing the operating state of the detection processing for the associated lesion.

4. The apparatus according to claim 3, wherein the information presentation unit discriminatively presents, on a display unit, detection results of a plurality of lesions detected by the lesion detection unit and the detection result of the associated lesion.

5. The apparatus according to claim 3, wherein the information presentation unit presents detection results of a plurality of lesions and the information representing the operating state of the detection processing for each lesion together.

6. The apparatus according to claim 3, wherein the information presentation unit presents the detection result of the associated lesion and the information representing the operating state of the detection processing for the associated lesion together.

7. The apparatus according to claim 3,. wherein the information presentation unit presents a position of at least one lesion on display of the medical image data.

8. The apparatus according to claim 1, wherein the information representing the operating state includes information corresponding to presence/absence of execution of the detection processing by the lesion detection unit.

9. The apparatus according to claim 1, wherein the information representing the operating state includes information representing that the detection processing by the lesion detection unit is being executed.

10. The apparatus according to claim 9,wherein
in a case where the detection processing is being executed, the operating state obtaining unit obtains, from the lesion detection unit, information representing a degree of progress of the detection processing and information representing a time remaining until an end of the detection processing, and
the information presentation unit presents at least one of the degree of progress of the detection processing and the time remaining until the end of the detection processing together with the information representing the operating state.

11. The apparatus according to claim 8, wherein the information representing the operating state includes information concerning whether the detection processing can be executed or not.

12. The apparatus according to claim 11, further comprising an instruction unit configured to instruct execution of the detection processing by the lesion detection unit.

13. The apparatus according to claim 12, wherein in a case where the detection processing is unexecuted, and the detection processing can be executed, the instruction unit instructs the lesion detection unit to execute the detection processing.

14. The apparatus according to claim 12, wherein the information corresponding to the reason includes information representing “unintroduced” in which a lesion detection function is not introduced to the lesion detection unit, and information representing that the medical image data is data outside an execution condition of the detection processing by the lesion detection unit.

15. The apparatus according to claim 14, wherein in a case where the information corresponding to the reason is the information representing “unintroduced”, the instruction unit instructs a lesion detection introduction unit to introduce an unintroduced lesion detection function.

16. The apparatus according to claim 14, wherein the information presentation unit presents an image obtaining confirmation window including a condition designation portion capable of adding or changing a condition to obtain medical image data that satisfies the execution condition of the detection processing.

17. The apparatus according to claim 14, wherein in a case where the information corresponding to the reason is the information representing that the medical image data is data outside the execution condition, the instruction unit instructs an image obtaining unit to obtain medical image data that satisfies the execution condition of the detection processing.

18. An information processing method comprising:
obtaining information representing an operating state of detection processing by a lesion detection unit configured to detect a lesion from medical image data: and
in a case where the information representing the operating state is information representing a state in which the detection processing cannot be executed, presenting information corresponding to a reason why the detection processing cannot be executed.

19. A non-transitory computer readable storage medium storing a program configured to cause a computer to function as an information processing apparatus defined in claim 1.
